(19) United States
(12) Patent Application Publication
(10) Pub. No.: US 2025/0376513 A1
(43) Pub. Date: Dec. 11, 2025

(54) USE OF ANTI-MCP1 NEUTRALIZING ANTIBODY IN PREPARING MEDICAMENT FOR TREATING SYSTEMIC INFLAMMATION CAUSED BY NEURODEGENERATIVE DISEASES

(71) Applicants: SHENZHEN INSTITUTES OF ADVANCED TECHNOLOGY CHINESE ACADEMY OF SCIENCES, Shenzhen (CN); SHENZHEN UNIVERSITY OF ADVANCED TECHNOLOGY, Shenzhen (CN)

(72) Inventors: Yu CHEN, Shenzhen (CN); Xueqi QU, Shenzhen (CN)

(73) Assignees: SHENZHEN INSTITUTES OF ADVANCED TECHNOLOGY CHINESE ACADEMY OF SCIENCES, Shenzhen (CN); SHENZHEN UNIVERSITY OF ADVANCED TECHNOLOGY, Shenzhen (CN)

(21) Appl. No.: 19/216,308

(22) Filed: May 22, 2025

Related U.S. Application Data
(63) Continuation of application No. PCT/CN2022/133518, filed on Nov. 22, 2022.

(51) Int. Cl. C07K 16/24; A61K 39/00; A61P 25/28; A61P 29/00; A61P 37/06

(52) U.S. Cl. CPC C07K 16/24; A61P 25/28; A61P 29/00; A61P 37/06; A61K 2039/54; A61K 2039/545; C07K 2317/76

(57) ABSTRACT

The present invention provides use of anti-MCP1 neutralizing antibody in preparing medicament for treating systemic inflammation caused by neurodegenerative diseases, and further provides use of the neutralizing antibody in preparing a medicament for treating central and peripheral inflammatory responses of neurodegenerative diseases. The neutralizing antibody can effectively inhibit systemic inflammatory responses caused by neurodegenerative diseases. The present invention provides a new way and orientation for treating neurodegenerative diseases.

### TECHNICAL FIELD

The present disclosure relates to the technical field of biomedicines, and relates to use of anti-MCP1 neutralizing antibody in preparing medicament for treating systemic inflammation caused by neurodegenerative diseases.

### BACKGROUND

Neurodegenerative diseases are chronic high-incidence diseases that are clinically manifested by a progressive decrease in behavioral, social, cognitive, or motor functions. The most common neurodegenerative diseases include Alzheimer's disease (AD), Parkinson's disease (PD), and the like. Research reports show that there were more than 50 million senile dementia patients worldwide in 2020, and it is estimated that there will be 152 million in 2050. As the population ages, the severity of the disease is becoming increasingly prominent.

The main histopathological manifestations of AD are the deposition of oligomeric β amyloid plaques (AB) and the formation of neurofibrillary tangles (NFT) by hyperphosphorylation of Tau protein. Studies have found that in addition to these classic features, the inflammatory response also plays an important role in the pathogenesis of AD. The long-term chronic inflammatory response induces neurodegeneration and leads to neuronal loss and even brain damage. There are common inflammatory response-inducing factors in most neurodegenerative diseases including AD, and these inducing factors can accelerate the development and progression of the diseases. Despite the anti-inflammatory treatment against these factors, the anti-inflammatory drugs currently used in clinical practice cannot effectively ameliorate neurodegenerative diseases and are at risk of causing autoimmunity.

Therefore, how to provide a drug based on peripheral minimally invasive or non-invasive administration, which is simple and convenient to operate, low in cost and safer to treat neurodegenerative diseases has become an urgent problem to be solved.

### SUMMARY

In view of the defects in the prior art and the actual need, the present disclosure aims to provide use of an anti-MCP1 neutralizing antibody in preparing a medicament for treating systemic inflammation caused by a neurodegenerative disease.

MCP1 is monocyte chemotactic protein-1, which has chemotactic activity for immune cells and induces immune cells to participate in inflammatory responses. Studies have shown that the MCP1 level in cerebrospinal fluid and plasma of AD patients is positively correlated with cognitive impairment of AD patients. In AD animal models, elevated MCP1 can aggravate Aβ amyloid plaque deposition, impaired neurogenesis, and cognitive dysfunction, suggesting that MCP1 plays an important role in the pathological progression of AD.

The technical solution adopted by the present disclosure is as follows:

In a first aspect, the present disclosure provides use of an anti-MCP1 neutralizing antibody in preparing a medicament for treating systemic inflammation caused by a neurodegenerative disease.

In a second aspect, the present disclosure provides use of an anti-MCP1 neutralizing antibody in preparing a medicament for treating central and peripheral inflammatory responses to a neurodegenerative disease.

Further, the neurodegenerative disease is Alzheimer's disease.

Further, the anti-MCP1 neutralizing antibody is used for: (1) inhibiting secretion of a peripheral chemokine MCP1; (2) inhibiting secretion of a peripheral inflammatory factor IL-6; (3) inhibiting activity of peripheral T lymphocytes; and (4) inhibiting infiltration of peripheral immune cells.

Further, the peripheral immune cell is a cytotoxic T cell.

Further, a safe and effective dose of the anti-MCP1 neutralizing antibody is 0.125-0.25 mg/kg, so as to ensure that the anti-MCP1 neutralizing antibody has the effect of neutralizing the chemokine MCP1 in vivo without causing toxic and side effects. The “safe and effective dose” refers to an amount of the antibody sufficient to significantly improve the condition without causing severe side effects.

Further, the route of administration of the anti-MCP1 neutralizing antibody is peripheral administration.

Further, the route of administration of the anti-MCP1 neutralizing antibody is minimally invasive or non-invasive administration.

Further, the route of administration of the anti-MCP1 neutralizing antibody includes, but is not limited to, intravenous injection.

Preferably, the cycle of injecting the antibody of the present disclosure includes, but is not limited to, two weeks of injection with injections given every other day; it may also be a single injection or continuous administration. Those skilled in the art can monitor the condition of the individual throughout the treatment process and adjust the injection cycle of the injected antibody of the present disclosure accordingly.

In a third aspect, the present disclosure provides a method for treating systemic inflammation caused by a neurodegenerative disease or treating central and peripheral inflammatory responses to a neurodegenerative disease, which comprises administering to a patient an anti-MCP1 neutralizing antibody by peripheral administration.

Further, the neurodegenerative disease is Alzheimer's disease.

Further, a safe and effective dose of the anti-MCP1 neutralizing antibody is 0.125-0.25 mg/kg.

Further, the peripheral administration includes, but is not limited to, intravenous injection.

Compared with the prior art, the present disclosure has the following beneficial effects:

The present disclosure finds that the anti-MCP1 neutralizing antibody can inhibit the secretion of the peripheral chemokine MCP1, inhibit the secretion of the peripheral inflammatory factor IL-6, inhibit the activity of peripheral cytotoxic T lymphocytes, and inhibit the infiltration of peripheral immune cells. The anti-MCP1 neutralizing antibody can inhibit the peripheral and central inflammatory responses, which has practical application significance for inhibiting the systemic inflammation caused by neurodegenerative diseases. Experiments prove that the anti-MCP1 neutralizing antibody can effectively inhibit the systemic inflammatory responses caused by neurodegenerative diseases. The present invention provides a new way and orientation for treating neurodegenerative diseases.

The anti-MCP1 neutralizing antibody of the present disclosure adopts a peripherally-based minimally invasive or non-invasive administration, is simple to operate, low in cost and risk, has no toxic and side effects, is easily accepted by patients, and is widely applicable. The present disclosure determines that the safe and effective dose of the anti-MCP1 neutralizing antibody is 0.125-0.25 mg/kg. The dose can not only ensure the maintenance of the therapeutic effect, but also reduce the toxic and side effects, which has practical application value, can be used for preparing an AD therapeutic drug or in combination with other AD therapeutic drugs, and has wide application prospect.

### BRIEF DESCRIPTION OF DRAWINGS

FIG. 1 shows the experimental design for the administration to animals in Examples 1˜4 of the present disclosure, wherein the administration is performed by intravenous injection in two-week treatment cycles with injections given every other day.

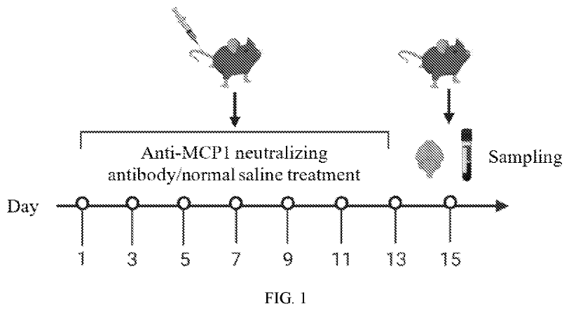

FIG. 2 shows the results of the changes in the expression level of MCP1 in the peripheral blood of wild-type mice and AD model mice treated with the anti-MCP1 neutralizing antibody or normal saline in Example 1 of the present disclosure.

FIG. 3 shows the results of the changes in the expression level of inflammatory factors in the peripheral blood of wild-type mice and AD model mice treated with the anti-MCP1 neutralizing antibody or normal saline in Example 2 of the present disclosure.

FIG. 4 shows the different proportions of cells in the gate of wild-type mice and AD model mice treated with the anti-MCP1 neutralizing antibody or normal saline in PBMC flow cytometry in Example 3 of the present disclosure.

FIG. 5 shows the immunofluorescence staining results of brain tissues of wild-type mice and AD model mice treated with the anti-MCP1 neutralizing antibody or normal saline in Example 4 of the present disclosure (scale bar=50 μm).

### DETAILED DESCRIPTION OF THE EMBODIMENTS

To further describe the technical means adopted by the present disclosure and the effects thereof, the present disclosure is further explained below with reference to examples and accompanying drawings. It should be understood that the specific embodiments described herein are merely intended to explain the present disclosure but not to limit the present disclosure.

The examples with no specified techniques or conditions are performed in accordance with techniques or conditions described in the literature in the art or in accordance with the product instructions. The reagents or instruments provided with no manufacturer specified are conventional and commercially available products.

### Material:

Wild-type mouse and AD mouse models were from Jackson Laboratory, USA.

The anti-MCP1 neutralizing antibody was purchased from BD Biosciences, Cat. No. 5554440.

Paraformaldehyde was purchased from Sigma-aldrich, Cat. No. 158127.

The embedding agent OCT was purchased from SAKURA, Cat. No. 4583.

The CD8 primary antibody was purchased from Invitrogen, Cat. No. 14-0195-82.

The fluorescent secondary antibody was purchased from Thermo Scientific.

The red blood cell lysis buffer was purchased from BD Biosciences, Cat. No. 555899.

The antibody used in the flow cytometry was purchased from BD Biosciences, with Cat. Nos.:
Ms CD45 FITC 30-F11, Cat. No. 553079;
Ms CD3 MolCpx PerCP-Cy5.5 17A2, Cat. No. 560527;
Ms CD4 APC-H7 GK1.5, Cat. No. 560181;
Ms CD8a PE 53-6.7, Cat. No. 553032.

Horse serum was purchased from Gibco, Cat. No. 26050088.

Fetal bovine serum was purchased from Life Technologies, Cat. No. 16050-122.

DAPI was purchased from Thermo Scientific, Cat. No. D1306.

DPBS was purchased from Sigma, Cat. No. D8662-24*500ML.

Trtion-X100 was purchased from Sigma Aldrich, Cat. No. X100-500ML.

ELISA kit (MCP1) was purchased from R&D, Cat. No. DY479-05.

ELISA kit (IL-6) was purchased from R&D, Cat. No. M6000B.

### Example 1

In this example, the ELISA kit was used to detect the expression level of MCP1 in the peripheral blood of wild-type mice and AD model mice separately treated with the anti-MCP1 neutralizing antibody or normal saline, and the steps were as follows:

### 1. Sample Collection

### (1) Serum Sample Collection:

Wild-type mice and AD model mice treated with the anti-MCP1 neutralizing antibody or normal saline were anesthetized with isoflurane gas, and blood samples of the mice were collected through the fundus into 1.5 mL sterile EP tubes. The mice were sacrificed by cervical dislocation and rapid decapitation with scissors, and serum was isolated according to the following procedures:

The anticoagulant samples were left to stand at 4° C. for 4 h, and serum was naturally precipitated after the blood was coagulated. The sample was centrifuged at 4000 rpm for 30 min at 4° C. to separate the serum, and the insoluble substance was discarded.

The serum was transferred to a new sterilized EP tube, aliquoted and stored at −80° C. for later use.

### 2. Preparation of Reagents:

(1) Preparation of positive control: The MCP1 positive control was dissolved in 1 mL of deionized water and well mixed for later use.
(2) Preparation of washing solution: The working concentration was achieved by dilution with deionized water at 1:25.
(3) Preparation of luminescent reagent: 15 min before detection on a machine, the luminescent reagent A and reagent B in the kit were mixed at a volume ratio of 1:1 and stored in the dark.
(4) Preparation of MCP1 standard: The 5000 μg/mL standard provided in the kit was diluted in a new EP tube to obtain a 500 μg/mL standard at a ratio of 1:10 with a calibration diluent, which was then separately diluted to obtain standards with concentrations of 250 μg/mL, 125 μg/mL, 62.5 pg/mL, 31.3 pg/mL, 15.6 pg/mL, and 7.81 pg/mL.

### 3. ELISA Assay

(1) 100 μL of assay diluent RD1W was added to the test wells.
(2) A standard, a control sample, and a test sample were sequentially added to the test wells, and the plate was sealed with a sealing film provided in the kit and then incubated at room temperature for 2 h.
(3) After the incubation was completed, the sealing film was removed, and the liquid was discarded. The assay plate was washed 4 times with 400 μL of the washing solution in each well, and the washing solution was discarded.
(4) 100 μL of mouse MCP1 conjugate was added to the test wells, and the plate was sealed with a sealing film and then incubated at room temperature for 2 h.
(5) Step (3) was repeated once.
(6) 100 μL of the prepared luminescent reagent was added to the test wells, and the plate was incubated at room temperature for 20 min in the dark.
(7) 50 μL of a stop solution was added, and the content in the assay plate was gently mixed to ensure thorough mixing.
(8) Plate reading: The optical density of each well was measured within 30 min.
(9) Calculation: Quantitative calculation of concentrations was performed according to the formula provided in the kit.

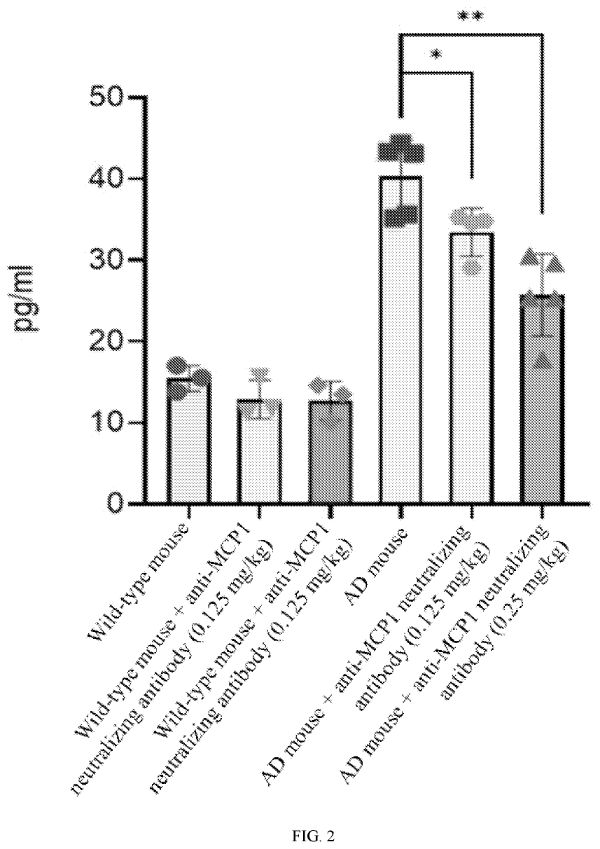

The assay results are shown in FIG. 2.

As can be seen from FIG. 2, after the wild-type mice were treated with the anti-MCP1 neutralizing antibody, the expression level of MCP1 in peripheral blood was not significantly different from that of the normal saline injection group. The expression level of MCP1 in the peripheral blood of the AD model mice treated with the anti-MCP1 neutralizing antibody was significantly down-regulated as compared to the normal saline injection group. The above results show that the anti-MCP1 neutralizing antibody of the present disclosure has a neutralizing effect in mice, and the administration regimen of the present disclosure does not cause excessive immune response, suggesting a low application risk.

### Example 2

In this example, the ELISA kit was used to detect the expression level of IL-6 in the peripheral blood of wild-type mice and AD model mice separately treated with the anti-MCP1 neutralizing antibody or normal saline, and the steps were as follows:

### 1. Sample Collection

### (1) Serum Sample Collection:

Wild-type mice and AD model mice treated with the anti-MCP1 neutralizing antibody or normal saline were anesthetized with isoflurane gas, and blood samples of the mice were collected through the fundus into 1.5 mL sterile EP tubes. The mice were sacrificed by cervical dislocation and rapid decapitation with scissors, and serum was isolated according to the following procedures:

The anticoagulant samples were left to stand at 4° C. for 4 h, and serum was naturally precipitated after the blood was coagulated. The sample was centrifuged at 4000 rpm for 30 min at 4° C. to separate the serum, and the insoluble substance was discarded.

The serum was transferred to a new sterilized EP tube, aliquoted and stored at −80° C. for later use.

### 2. Preparation of Reagents:

(1) Preparation of positive control: The IL-6 positive control was dissolved in 1 mL of deionized water and well mixed for later use.
(2) Preparation of washing solution: The working concentration was achieved by dilution with deionized water at 1:25.
(3) Preparation of luminescent reagent: 15 min before detection on a machine, the luminescent reagent A and reagent B in the kit were mixed at a volume ratio of 1:1 and stored in the dark.
(4) Preparation of IL-6 standard: The 5000 μg/mL standard provided in the kit was diluted in a new EP tube to obtain a 500 μg/mL standard at a ratio of 1:10 with a calibration diluent, which was then separately diluted to obtain standards with concentrations of 250 μg/mL, 125 μg/mL, 62.5 pg/mL, 31.3 pg/mL, 15.6 pg/mL, and 7.81 pg/mL.

### 3. ELISA Assay

(1) 100 μL of assay diluent RD1W was added to the test wells.
(2) A standard, a control sample, and a test sample were sequentially added to the test wells, and the plate was sealed with a sealing film provided in the kit and then incubated at room temperature for 2 h.
(3) After the incubation was completed, the sealing film was removed, and the liquid was discarded. The assay plate was washed 4 times with 400 μL of the washing solution in each well, and the washing solution was discarded.
(4) 100 μL of mouse IL-6 conjugate was added to the test wells, and the plate was sealed with a sealing film and then incubated at room temperature for 2 h.
(5) Step (3) was repeated once.
(6) 100 μL of the prepared luminescent reagent was added to the test wells, and the plate was incubated at room temperature for 30 min in the dark.
(7) 100 μL of a stop solution was added, and the content in the assay plate was gently mixed to ensure thorough mixing.
(8) Plate reading: The optical density of each well was measured within 30 min.
(9) Calculation: Quantitative calculation of concentrations was performed according to the formula provided in the kit.

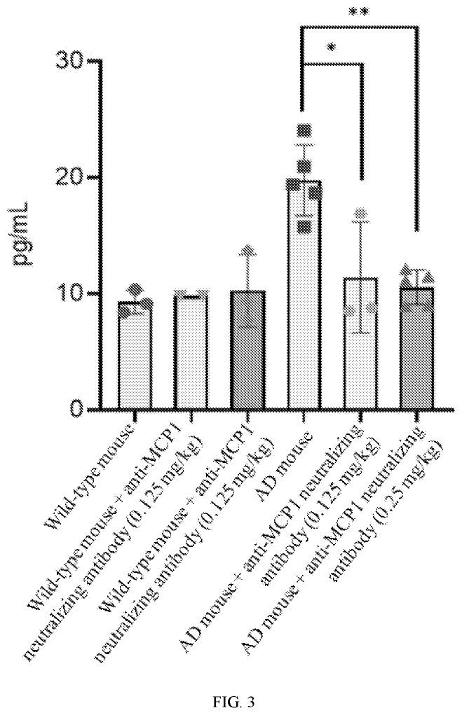

The assay results are shown in FIG. 3.

As can be seen from FIG. 3, after the wild-type mice were treated with the anti-MCP1 neutralizing antibody, the expression level of IL-6 in peripheral blood was not significantly different from that of the normal saline injection group. The expression level of IL-6 in the peripheral blood of the AD model mice treated with the anti-MCP1 neutralizing antibody was significantly down-regulated as compared to the normal saline injection group. The above results show that the anti-MCP1 neutralizing antibody of the present disclosure can down-regulate the peripheral immunoinflammatory response caused by AD.

### Example 3

In this example, the peripheral blood mononuclear cells (PBMCs) of wild-type mice and AD model mice separately treated with the anti-MCP1 neutralizing antibody or normal saline were subjected to flow cytometry analysis, and the steps were as follows:

### 1. Preparation of Highly Active Peripheral Blood Mononuclear Cells (PBMCs)

(1) To 200 μL of anticoagulated whole blood was added 3 volumes of red blood cell lysis buffer, and the mixture was gently mixed well and left to stand at room temperature for 10 min, during which the mixture was gently mixed well twice to lyse the red blood cells.
(2) The mixture was centrifuged at 800×g for 2 min, the supernatant was discarded, the cells were collected, and the sample was washed once with 1 mL of PBS.
(3) The antibodies were resuspended in 500 μL of buffer, filtered through a 300-mesh cell filter screen, incubated, and analyzed on a machine.

### 2. Flow Cytometry

(1) The cell suspension of PBMCs prepared in the above steps was adjusted to a cell density of 5×10⁶ cells/mL with DPBS containing 2% fetal bovine serum.
(2) 40 μL of the cell suspension was added into a plastic centrifuge tube containing 50 μL of fluorescently labeled specific antibody in advance, and then 50 μL of inactivated normal horse serum (diluted with DPBS at a ratio of 1:20) was added. The mixture was incubated at 4° C. for 30 min.
(3) 2 mL of DPBS containing 2% fetal bovine serum was added, and the cells were suspended and well mixed. The cells were washed and centrifuged at 1000 rpm at 4° C. for 5 min, and the washing was repeated once.
(4) 500 μL of pre-cooled PBS was added to resuspend the cells, and the mixture was ready for analysis.

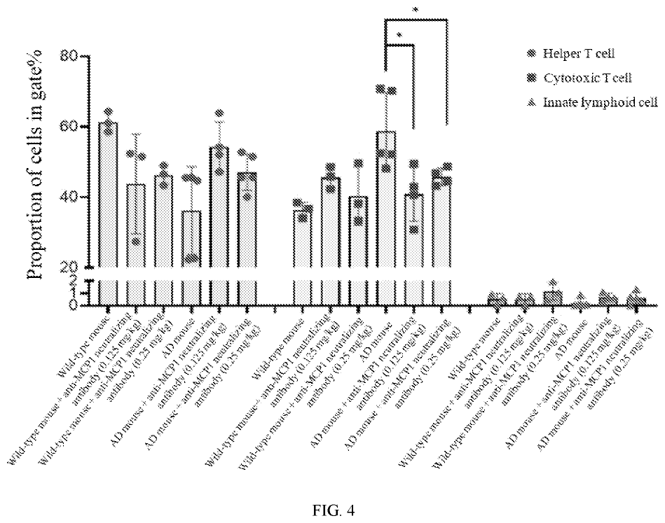

The proportions of cytotoxic T (Tc) cells, helper T (Th) cells, and innate lymphoid cells (ILCs) in different cell phyla were separately counted, and the statistical results are shown in FIG. 4.

As can be seen from the figure, after the wild-type mice were treated with the anti-MCP1 neutralizing antibody, there was no significant change in the activity of peripheral cytotoxic T (Tc) cells as compared to the normal saline treatment group. The activity of peripheral cytotoxic T (Tc) cells of the AD model mice treated with the anti-MCP1 neutralizing antibody was significantly down-regulated as compared to the normal saline injection group. The above results show that the anti-MCP1 neutralizing antibody of the present disclosure can down-regulate the peripheral immunoinflammatory response caused by AD, and the administration regimen of the present disclosure does not cause excessive immune response, suggesting a low application risk.

### Example 4

In this example, an immunofluorescence staining kit was used to stain CD8 on brain tissues of wild-type mice and AD mouse models treated with the anti-MCP1 neutralizing antibody or normal saline, and the steps were as follows:

### 1. Mouse Brain Tissue Section

(1) Anesthesia and brain tissue perfusion sampling: The mice were anesthetized by intraperitoneal injection of chloral hydrate. After deep anesthesia, the mice were fixed on a surgical board and placed in a dissection tray. The whole brains were collected by decapitation and fixed by soaking in paraformaldehyde for 24 h.
(2) Perfusion and fixation of mice: The mice were perfused with PBS at 4° C. at 20 mL/mouse, and then perfused with 4% paraformaldehyde at 4° C. (40 g of paraformaldehyde was weighed out and dissolved in a glass container containing 500 mL of DEPC water, and the mixture was continuously heated and magnetically stirred to 60° C. to form a milky-white suspension; the pH was adjusted to 7.0 with 1.0 mmol/L NaOH to make the solution clear; about 500 mL of 2×PBS was added, and the mixture was mixed well and filtered; the volume was made up to 1000 mL, and the mixture was stored at 4° C. for later use) at 20 mL/mouse until the tissue became hard.
(3) Sampling: The brain tissue was carefully removed, placed in a 15 mL centrifuge tube, and fixed with 4% paraformaldehyde (fixing agent) for 24 h.
(4) Dehydration: The tissue fixed with paraformaldehyde was washed 3 times with PBS (cleaning solution), dehydrated with 20% sucrose (dehydrating agent) until the tissue settled to the bottom, and dehydrated with 30% sucrose at 4° C. overnight.
(5) The embedding agent OCT was added dropwise to the specimen stage, put into a cryostat microtome until it became white, and then taken out, and the surface was quickly trimmed with a single-sided blade.
(6) The specimen bottom was cut flat with a safety blade and then adhered to the specimen stage. The specimen stage was then placed in the freezing stage of a −24° C. cryostat microtome. When the tissue was slightly whitish, a thin layer was coated on the specimen surface with OCT. The specimen was frozen for another 20 min.
(7) After the section thickness was adjusted, the sections were cut to a thickness of 40 μm. The cut sections were continuously collected and transferred to a 24-well plate containing PBS.
(8) The cut pieces were stored at 4° C. for later use.

### 2. Immunofluorescence Staining

(1) Sections at appropriate positions were carefully transferred into a 24-well plate containing 1 mL of pre-cooled PBS in advance, and washed 3 times with pre-cooled PBS, 10 min each time.
(2) Puncturing and blocking: 0.2% Triton X-100 (diluted with PBS), 0.1% BSA, and 5% horse serum (diluted with PBS) were added and incubated at room temperature for 40 min, and then placed on a shaker with slow shaking.
(3) The plate was washed with PBS 3 times at room temperature, each time for 5 min.
(4) Primary antibody incubation: The dilution was performed using the antibody dilution (PBS containing 0.01% BSA and 5% horse serum) at 1:100, 200 μL was added to each well, and the plate was incubated overnight at 4° C. with slow shaking.
(5) The primary antibody was recovered and washed with PBS 3 times at room temperature, each time for 10 min.
(6) The brain slices were blocked with 3% horse serum at room temperature for 30 min.
(7) Secondary antibody incubation and DAPI staining: The secondary antibody was diluted in a 1:500 ratio with PBS, and incubated at room temperature for 2 h in the dark; the DAPI stock solution was diluted in a 1:5000 ratio, and incubated at room temperature for 15 min.
(8) The plate was washed with PBS 3 times at room temperature, each time for 15 min.
(9) Sealing: A viscous glass slide was taken, and specific information was marked by a pencil on the right frosted surface. A drop of PBS was added in the middle of the glass slide, and the section was picked up and placed on the PBS drop. The PBS solution was removed by pipetting, 160 μL of a mounting medium was horizontally placed in the center of the glass slide, and the section was covered with a long cover slide to avoid bubbles and wrinkles.
(10) The glass slide was placed flat in the dark and dried.

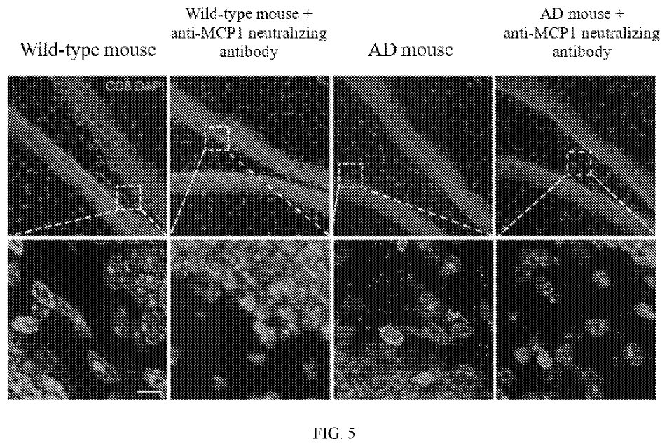

Pictures of the staining of wild-type mice and AD model mice treated with the anti-MCP1 neutralizing antibody or normal saline are shown in FIG. 5. As can be seen from the figure, the expression levels of cytotoxic T (Tc) cells in the brains of the AD model mice treated with the anti-MCP1 neutralizing antibody were significantly down-regulated as compared to the normal saline injection group. The above results show that the anti-MCP1 neutralizing antibody of the present disclosure can down-regulate the infiltration of peripheral immune cells caused by AD.

In conclusion, the anti-MCP1 neutralizing antibody described herein can neutralize the secretion of peripheral MCP1, inhibit the secretion of peripheral inflammatory factor IL-6, regulate the activity of peripheral T lymphocytes, and inhibit the infiltration of peripheral immune cells. The treatment method of the present disclosure can ensure that the antibody has a neutralizing effect in vivo without causing toxic and side effects. Therefore, the anti-MCP1 neutralizing antibody has practical significance for inhibiting the inflammatory response to neurodegenerative diseases.

The applicant declares that in the present disclosure, the examples above are used to describe the detailed method of the present disclosure, but the present disclosure is not limited to the detailed method above, that is, it does not mean that the present disclosure must rely on the detailed method above to be carried out. It will be appreciated by those skilled in the art that any modifications to the present disclosure, equivalent substitutions of the starting materials of the product of the present disclosure and the addition of auxiliary components, selections of specific modes, etc., are within the scope and disclosure of the present disclosure.

What is claimed is:

1. Use of an anti-MCP1 neutralizing antibody in preparing a medicament for treating systemic inflammation caused by a neurodegenerative disease or an anti-MCP1 neutralizing antibody in preparing a medicament for treating central and peripheral inflammatory responses to a neurodegenerative disease.

2. The use according to claim 1, wherein the neurodegenerative disease is Alzheimer's disease.

3. The use according to claim 1, wherein the anti-MCP1 neutralizing antibody is used for: (1) inhibiting secretion of a peripheral chemokine MCP1; (2) inhibiting secretion of a peripheral inflammatory factor IL-6; (3) inhibiting activity of peripheral T lymphocytes; and (4) inhibiting infiltration of peripheral immune cells.

4. The use according to claim 3, wherein the peripheral immune cell is a cytotoxic T cell.

5. The use according to claim 1, wherein a safe and effective dose of the anti-MCP1 neutralizing antibody is 0.125-0.25 mg/kg.

6. The use according to claim 1, wherein the route of administration of the anti-MCP1 neutralizing antibody is peripheral administration.

7. The use according to claim 6, wherein the route of administration of the anti-MCP1 neutralizing antibody is minimally invasive or non-invasive administration.

8. The use according to claim 7, wherein the route of administration of the anti-MCP1 neutralizing antibody includes, but is not limited to, intravenous injection.

9. A method for treating systemic inflammation caused by a neurodegenerative disease or treating central and peripheral inflammatory responses to a neurodegenerative disease, comprising administering to a patient an anti-MCP1 neutralizing antibody by peripheral administration.

10. The method according to claim 9, wherein the neurodegenerative disease is Alzheimer's disease.

11. The method according to claim 9, wherein a safe and effective dose of the anti-MCP1 neutralizing antibody is 0.125-0.25 mg/kg.

12. The method according to claim 9, wherein the peripheral administration includes, but is not limited to, intravenous injection.
